(19) United States
(12) Patent Application Publication
(10) Pub. No.: US 2005/0107711 A1
(43) Pub. Date: May 19, 2005

(54) Kind of method by the network hospital of diagnosing disease and re-segmentation type with the wrist radius artery blood vessel pulsing information

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/713,703

(22) Filed: Nov. 17, 2003

(51) Int. Cl. A61B 5/02

(52) U.S. Cl. 600/500

(57) ABSTRACT

The invention is a kind of medical diagnosis method. The invention use wrist radius artery digital indicator, after collecting 18 kinds of artery blood vessel pulse standard value, graphs on radius artery, edit them into standard pattern data base software, provides the user that after the user input the information from the digital indicator, the determined input information for consulting results will be the output, when the user browses on internet by distant landing, make the software and website data base connected together, to handle the processing continuously, decide the diagnostic of resegmenting type of specific disease and disease group of 18 kinds of radius artery blood vessel pulse standard value, the difference between the determined value and the standard value could reason out the specific diagnosing result.

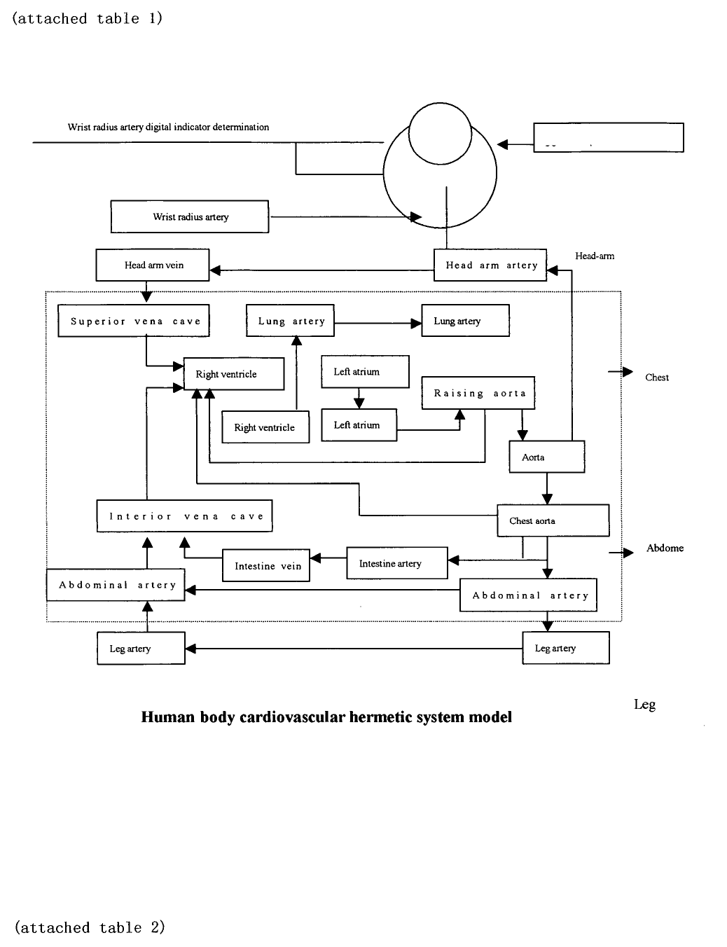

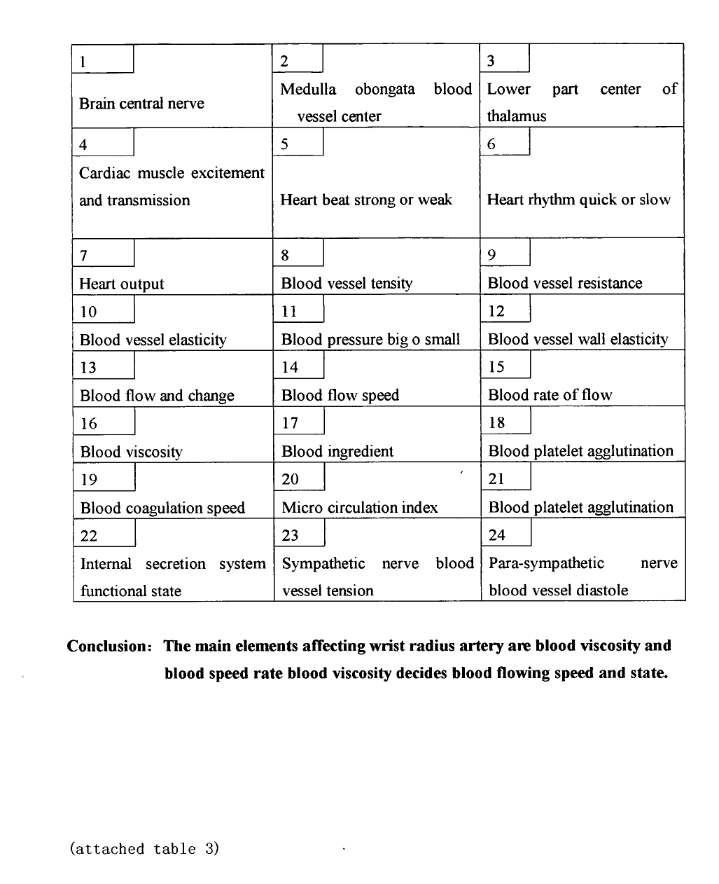

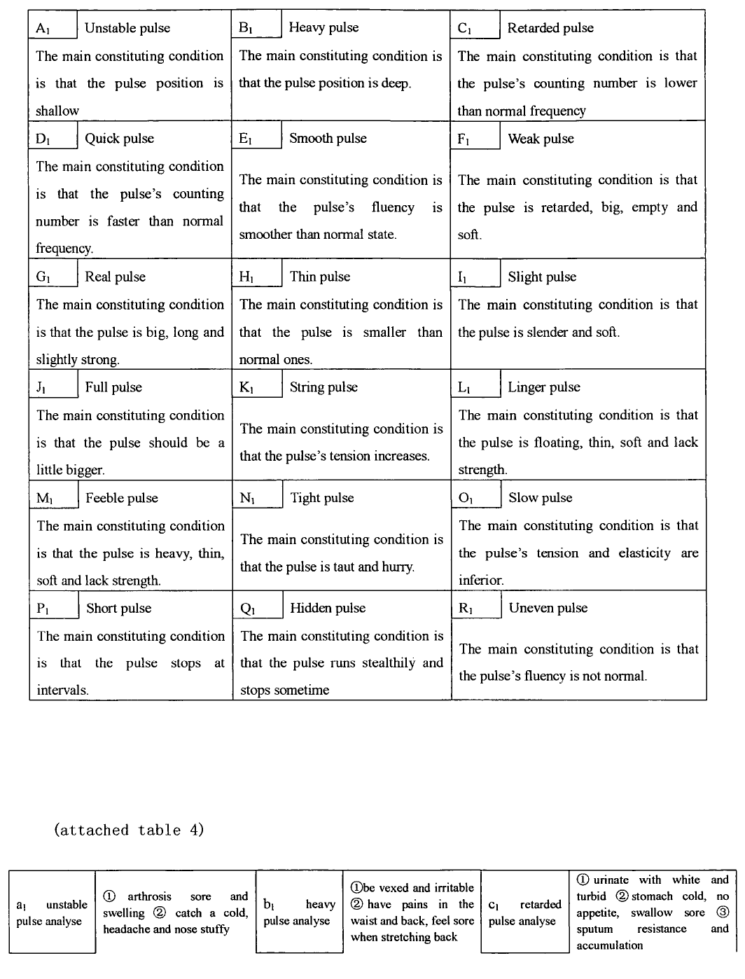

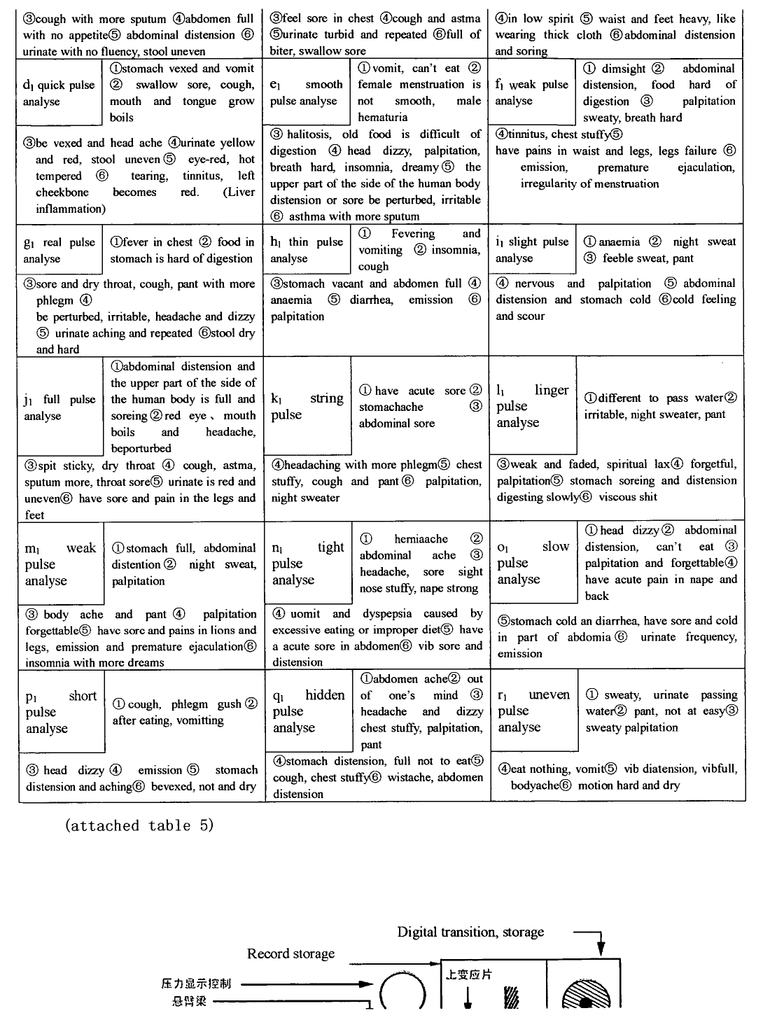

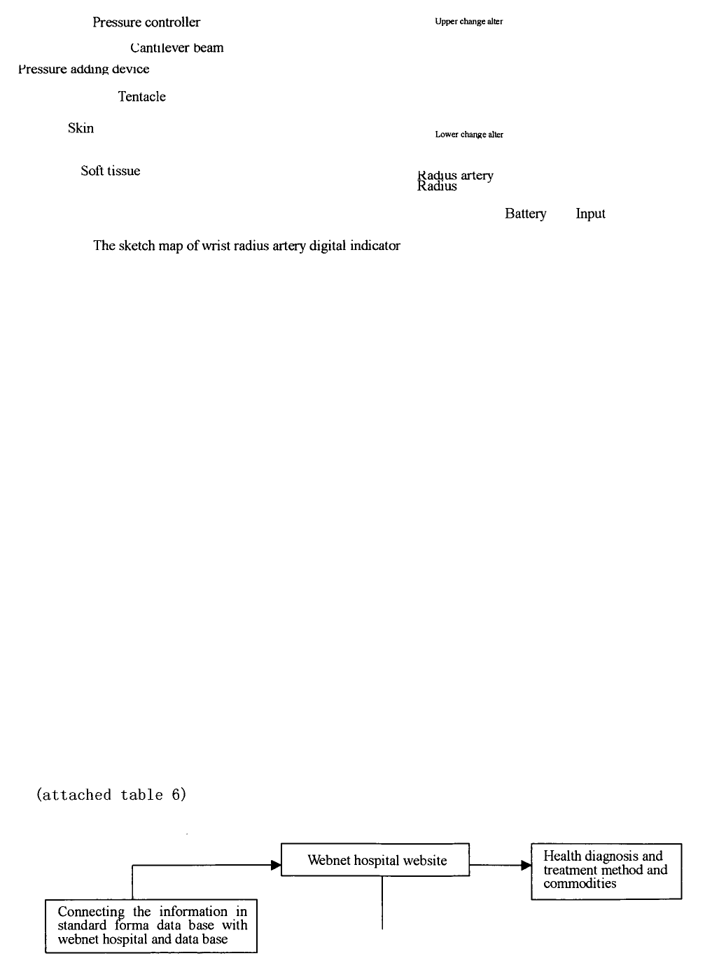

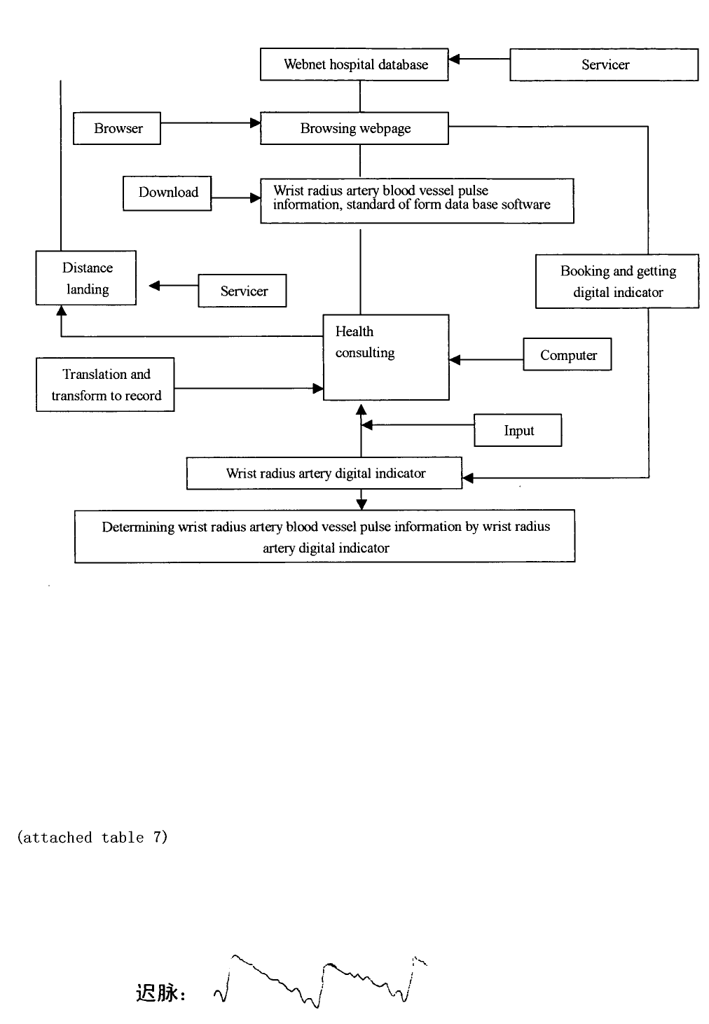

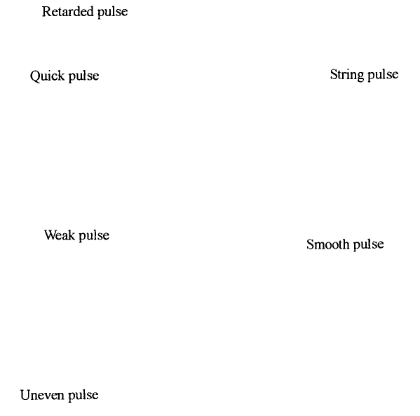

### BACKGROUND OF THE INVENTION

This patent is an invention of medicine diagnosis method, is a kind of diagnosis method by the network hospital of diagnosing disease and re-segmentation type with the wrist radius artery blood vessel pulsing information through wrist radius artery digital indicator. It uses digital indicator to record case history, auscultate the different pants of body, undertake lots of health information checkup and technical examination and test through wrist radius artery digital indicator, edit the information into logical and linear 18 standard value pictures, and store the information into the software of data base. Comparing the patient's radius artery blood vessel pulsing information tested by the digital indicator with the standard value pictures and data stored in the software of data base to analyze the difference, use the compared difference as the specific reasoning medical treatment diagnosis results of diagnosing disease and re-segmentation type through wrist radius artery blood vessel information, and can get the specific clue of medical treatment and specific diagnosis and treatment plan. It really reflects the patient's morbid state, it has realized intellectually and individually healthy medical treatment diagnosis and curing service. No. 101 clause in American Patent law stipulates that anyone who invents or discovers any novel and practical method, machine, product or compound, or their novel and practical improvement may gain patent according to its requirement and condition.

In the case of OJ1988, 308T, 385/86, the appeal committee thinks that a kind of diagnosis method should include not only the analyzing method for the specific results, but also the diagnosis from the analyzing results. The appeal committee thinks diagnosis method in No. 52 clause of the convention means that testing for the real value and the diagnostic determination on the base of testing, namely, the difference between testing value and standard value, all must undertake on live human body and animal body. It includes recording case history, observing, feeling, auscultating the parts of body, undertaking lots of health checkup and technical examination and testing, checking and material collecting period. He also includes the period for comparing testing value with standard value, recording any meaningful difference diagnostic, finding out the specific diagnosis results for the difference and realizing reasoning medical treatment decision. The appeal committee advances that there would be no diagnosis method existing without any part of the above mentioned three steps, it would at most be the method for obtaining the data applying in diagnosis method or data processing method. The appeal committee makes further comments that it is not sufficient to undertake the state checking on human body or animal body simply for the purpose of medical treatment. The determined situation must be alble to reflect the difference of morbid state itself. Testing the blood pressure is an absolute value which can only show the difference compared with standard value. Only the difference from a certain disease or a group of diseases by comparing their features and clear implication can make the relative testing a diagnosis method. The explanation and further comments made by the appeal committee are the most accurate annotation and appraise for the diagnosis method of diagnosing disease and re-segment type with wrist radius artery blood vessel pulsing information, the also clearly annotate the essential differences between the patent and the method for blood pressure testing.

The patent is a diagnosis method invented on the basis of information theory. So-called information, namely, the signals that human being fell all the changes from the surrounding and human body by our feeling organs or apparatus, information theory is the theory built up on the basis of information. Hatlai published the “information transmission” in 1928, for the first time, advanced that information is code, symbol and alignment, not the content itself, it got rid of the positive elements and made a breakthrough of concept. In 1948, “Shennong” published “communicating mathematics theory”. It announced the creation of information theory, which is the basic theory for studying information resource, information place, information channel and coding. The achievement of electronic information theory has brought the hopeful light for many sciences. People tried to solve those difficult problems within sense of hearing, nerveology, physiology and psychology. In 1960s, information theory had important development, the main studying points are information and information resource coding problem. It was Ashby in U.S.A. who promoted information theory is a branch of combination theory, its essence is basically a number. In 1965, after charld put forward fuzzy mathematics, someone create of information concept on the basis of fuzzy collective theory, further more to try to set up fuzzy information theory. Halian scholar, Langgao pointed out in publication “information theory, new orientation and unsolved problems” in 1975, that the matters once eliminated cautiously in “Shennong” contribution, now are included in. This patent is the innovation and breakthrough of information theory in medicine and medical diagnosis science.

In clinical treatment, there are always some patients who fell flustered, stuffy, palpitated, pant, there are also some other patients who often have diagnostic of getting angry, but the results show that they are normal after the examinations by all kinds medical instruments such as electro cardiogram. B-ultrasonic, CT, chemical examination and coronary arteries (veins) radiography American medicine must also complete the transition from collecting macro-information to collecting micro-information. Information message and news, are also signal and symbols. The pictures and digital information of wrist radius artery blood vessel pulse mainly reflect the physiologic and pathologic phenomenon in cardiovascular system, internal secretion and nervous system, they are quantitative and numerical information. Reflecting the organic physiologic and pathologic changes through radius artery, collecting those information with rich content and integration by wrist artery blood vessel digital indicator, contrasting the differences within the testing value and the standard value, provide the specific disease resegment diagnosis and treating plans.

### BRIEF SUMMARY OF THE INVENTION

Medicine development has had close relationship with the scientific and technical development, now high-technology develops very fast, rising borderline science has been continually permeating into the medical field, life science has developed from macro-determination the nature to micro fixed quantity, medicine will walk into the new era of mathematical medicine with the digital age in 21 century. Radius artery blood vessel blood flowing dynamics is just the burgeoning science stating quantitatively the numerical control relationship within blood vessel blood flowing the central nervous system, internal secretion system and plant nervous system dynamics and which control the flowing dynamics in blood vessel. This patent is just the leader and typical representative in the research and development of the rising subject.

Human body is an organic, comparative closed circulatory. Wrist radius artery is the end of this comparative closing whole, is perfect organic information recourses, and also a collecting point for the whole information. This patent uses wrist radius artery digital indicator to input the accurately obtained data information at the end of radius artery and out put the consulting results of the tested value information, realizes storing and dealing with those data, and answer the inquires from customer end, operates continually.

The signals reflected by wrist radius artery blood vessel pulse show the relationship among the blood circulatory; plant nerve and internal secretion system, which have their own information system to handle their own information. Because all the organs, tissues in the organic have their method to gain, utilize, keep and transmit the information. In normal condition, information transmission among the different parts has enough reliability, the adjustment and control information among them all could be received by relative part and then makes reaction. The artery blood pressure adjustment of a normal person is also a kind of reaction adjustment. Organic tissues all have their own information system, which gather into central nerve, the adjust center of the central nerve reacts, so, all the information system have the function to use, keep and transmit the reacting information which unite all the parts of organic whole together to form a whole to keep organic stable. The whole set of information organic state reflects in the database through its important end, wrist radius artery blood vessel pulse. The network hospital diagnosis method of diagnosing disease and resegment type through wrist radius artery blood vessel pulsing information is just using wrist radius artery digital indicator to seek valuable end radius artery information to formalize its independent medical diagnosis method.

### BRIEF DESCRIPTION OF THE SEVERAL VIEWS OF THE DRAWING

Table 1 The principle for testing the pulsing information of wrist radius artery blood vessel to diagnose disease and resegment by wrist radius artery digital indicator.

Table 2 The main elements affecting the pictures and results of wrist radius artery blood vessel pulsing information tested by wrist radius artery digital indicator.

Table 3 The menu of the standard results of wrist radius artery blood vessel pulse tested by wrist radius artery digital indicator.

Table 4 Analyzing the wrist radius artery blood vessel pulse standard results menu to diagnose disease and re-segment.

Table 5 Wrist radius artery digital indicator and its function description.

Table 6 The real flow diagram for the medical diagnosis method of diagnosing disease and resegmenting according to the blood vessel pulse information of wrist radius artery tested by wrist radius artery digital indicator.

Table 7 The patent's wrist radius artery blood vessel pulse information really tested by wrist radius artery digital indicator and the illustrations for diagnosing patient's disease and resegmenting according to the standard pictures.

Table 1 The principle of diagnosing disease and resegmenting type with wrist radius artery blood vessel pulse information tested by wrist radius artery digital indicator. (see attcached table 1)

Table 2 The main elements affecting the graphs hand results of wrist radius artery blood vessel pulse information tested by wrist radius artery digital indicator. (see attcached table 2)

Table 3 The standard result menu of wrist rudius artery blood vessel pulse tested by wrist radius artery digital indicator (see attcached table 3)

Table 4 The menu difference and analyse for the standard results of wrist radius artery blood vessel pulse to diagnose resegment type disease (see attcached table 4)

Table 5 Wrist radius artery digital indictor and its function Description (see attcached table 5)

The function of wrist radius artery digital indicator:

Determining the pressure signal of general pulse dynamics of the pulse's fluency, rhythm, frequency, power, width and length in wrist radius artery. The signal could be transformed into number code storage through digital transformer, input into the computer when being used.

Table 6 The real flow diagram of the medical diagnosis method of diagnosing disease and resegmental type with the wrist artery blood vessel pulse information tested by wrist artery digital indicator. (see attcached table 6)

Table 7 The graphs of wrist radius artery blood vessel pulse information determined practically by wrist radius artery digital indicator and the real illustrations for diagnosing patients' disease and resegment type according to the standard graphs. (see attcached table 7)

### EXAMPLES

Retarded pulse: sinus brady-cardia

Quick pulse: sinus tachy cardia

String pulse: high-pressure caused by the increase of heart output and blood vessel resistance

Smooth pulse: gestation

Weak pulse: composite symptom for lack of nutrition

Uneven pulse: neurasthenia symptom

### DETAILED DESCRIPTION OF THE INVENTION

1. “The network hospital method of diagnosing disease and resegmental type through wrist radius artery blood vessel pulse information” is diagnosis method invention patent. The invention uses wrist radius artery digital indicator, with the assistance of professional acupuncturist, collects massively human body 18 kinds of standard radius artery blood vessel pulsing information pictures and standard data, organizes the data with the way of digital transition, completes logical system coding after building up immense data base software of radius artery blood vessel pulse information logical system, and then manufactures and sent freely a lot of wrist radius artery digital indicator to network hospital membership, starts the medical activities like health consultation, diagnosis and treatment, buying commodities with computers. Wrist radius artery is one of the most important, sensitive and reliable information resources of human activities. The information of radius artery pulse is a testable biological signal in the developing process of internal secretion system and nervous system within human circulatory system, the graphs formed with those human body valuable signal by recording according to its order with the same pressure are radius artery blood vessel pulsing graph.

2. Human body is an organic whole, its blood circulatory systems are close series and mutual establish ties system, wrist radius artery is the end of the whole close system (table 1), the patent is an innovation to the clinical medical system collecting radius artery blood vessel pulse information at the end of the circulation by using wrist radius artery digital indicator. Wrist radius artery blood vessel shows the function and affection of heart functional state, blood vessel functional state and blood's quality and quantity. It also reflects the functional state of plant nervous system and internal secretion system. Because of circulatory system, the affection could be read out indirectly in the radius artery pulse information. It also shows the functional states of organic plant nerve, internal secretion system and brain central system (Table 2). Collection and analysis of wrist radius artery blood vessel pulse information provide important illustrations for the characters of many kinds of disease, resegmental type and developing trends.

3. The patent invents and uses wrist radius artery digital indicator, which consists of tentacle, pressure-adding device, pressure meter, controlling device, upper and lower change alter, resistance voltage signal record, storing device, digital transition storing device, input connection device and battery (table 5). Its function is to test the voltage signal of the general pulsing dynamics of the fluent degree, rhythm, frequencies, power, width and length in wrist radius artery blood vessel. To translation the signal into number code through digital transformer and to input them into the computer. The way for using wrist radius artery digital indicator is: examinee lies on the bed, clam and relaxed, breathing calmly upper limbs stretching, the center of the palm upward, leaves wrist exposed, to be touched with finger for the pulsing apparently place, placing the center of indicator tentacle film aiming at the artery vessel, press the indicator's wrist ring structure and fasten the indicator. Press down the pressure-adding function button, waiting for it reaching to 100 mm Hg till it is stable at the value, and the information of wrist radius artery blood vessel pulse is in stable state, the indicator lamp at the side of the indicator window gives off yellow lights, the indicator starts work automatically. After collecting 4 sets of ware pattern of radius artery blood vessel pulse information, to turn off the button with single tough. Animal experiments show that the radius artery blood vessel pulsing information and graph curves gained from internal radius artery by inserting into the artery vessel with wound and from the radius artery surface by using wrist radius artery digital indicator, the parameters for calculating blood flowing dynamics are basically the same, its relativity is good, it indicts that the study for on-wound blood flowing dynamics parameter and the diagnosis method for human body's state from the pulse of radius artery by using wrist radius artery digital indicator has great future for its usage of development. Radius artery blood vessel carries a great deal of human body circulatory dynamics information.

4. The wrist radius artery blood vessel pulse directly comes from heart without passing heart cavity and other soft tissue, it also is not affected by the blood flow in heat cavity. The integrated cardiac cycle directly runs into the pulsing information of radius artery may directly collect and receive radius artery blood vessel pulse information cure, Pulse Wave Care by wrist radius artery digital indicator. The Pulse Wave Cure of radius artery blood vessel pulse information are consisted of different cures (table 7), is a dynamics processing in cardiac cycle, and them transform into another dynamics. In the transforming process, all cures decline with the features of class index.

With using wrist radius artery digital indicator, 30 professional acupuncturists who had passed the examination about professional practitioners for acupuncture and moxibustion major in USA, arrange 1000 volunteer in USA into many groups, there are three persons in one group, collect 18 kinds of typical, standard wrist radius artery blood vessel pulse information graphs and division (table 3, table 4), through the software edition process from editing input, effective data, linear program for solution, results patternized, handle the logic data system elements of determined value into logic system, complete the software system of webnet hospitable method of diagnosing disease and resegmental type with wrist radius artery blood vessel pulse information, finish the applying system from input of the gained data determinating value information to 18 kinds of standard, typical radius artery blood vessel pulse information standard value graphs and digital codes, which have been stored in the data base of website, servicer and computers.

5. The patent begin to use the real flow diagram of specific reasoning medical treatment of diagnosing and resegment, with the information determined by wrist artery digital indicator, to find out comparing the difference between determinated value and standard value. The flow process is that patient browses the page, books and posts the data indicator, use the record and store health information in a regulated way, while browing the page, download the standardized pattern data base software of wrist radius artery blood vessel, input the information in the digital indicator, after transforming them, we may read on the screen the individual health consulting suggestion and health information. Distance landing webnet hospital website will connect the information in data base software of wrist radius artery blood vessel pulse with webnet hospital data base, input one's own health information gained by wrist radius artery digital indicator, and compare, analyze, sort out the difference between the determined value and standard value, calculate the specific diagnosing results through those difference, the health diagnosis and treatment method and commodities will be displayed on the screen of terminal computer.

6. With the webnet hospital diagnosing method of diagnosing disease and resegmental type with wrist radius artery blood vessel pulse information, the decided 18 kinds of radius artery pulse information themselves can show the morbid state's difference (table 3, table 4). This diagnosis method compares the specific features of a disease or a group of disease with the standard value to point out the specific diagnostic of disease resegment and the specific pathogens For example: a patient has a cute pain in chest, with the diagnostic like cough, chest stuffy, night sweat, after gaining the wrist radius artery digital indicator from the webnet hospital website and standard pattern data base software, through testing, the computer screen shows the diagnosis suggestion like blood viscosity high, the type of figure extra fat, and then distance landing website hospital website and go to the hospital to ask for diagnosis, examination, website and hospital made the diagnosis that the patient has the disease of high pressure because of output and blood vessel resistance incensement. Webnet hospital database take the determined value to compare with the standard value in the database, make sure its string pulse standard value (table 7). According to the main feature of artery blood vessel pulse tension increasing resegment it as blood viscosity high, pressure high, fatty, has the diagnostic of blood silted up.

1. I announce that “a webnet hospital method of diagnosing disease and resegmenting type with wrist radius artery blood vessel pulse information” is my invention.
I think my invention is examining human wrist radius artery blood vessel pulse by using wrist radius artery digital indicator and compiling 18 kinds of radius artery blood vessel pulse standard value, graphs, comparing the determined value through digital indicator with standard value, sort out the diagnostic of resegmenting type of specific disease and disease group of 18 kinds of radius artery blood vessel pulse standard value, the difference between the determined value and the standard value would give the reasoning medical specific diagnosis result.
I think that my invention in dudes the principle of wrist rudius artery blood vessel pulse information determined by wrist rudius artery digital indicator (table 1), standard result menu (table 3), standard result menu analyse table 4) m wrist rudius artery digital indicator and its function (table 5), real flow diagram (table 6) and wrist rudius artery blood vessel pulse information standard pattern data base software.
